(19) United States
(12) Patent Application Publication
(10) Pub. No.: US 2023/0059310 A1
(43) Pub. Date: Feb. 23, 2023

(54) PREDICTION SYSTEM, PREDICTION DEVICE, PREDICTION METHOD, AND NON-TRANSITORY COMPUTER READABLE STORAGE

(71) Applicant: JAPAN COMPUTER VISION CORP., Tokyo (JP)

(72) Inventors: Toshihiro UTSUMI, Tokyo (JP); Chikashi OKAMOTO, Tokyo (JP)

(73) Assignee: JAPAN COMPUTER VISION CORP., Tokyo (JP)

(21) Appl. No.: 17/886,806

(22) Filed: Aug. 12, 2022

(30) Foreign Application Priority Data
Aug. 19, 2021 (JP) 2021-134364

(51) Int. Cl. A61B 5/00; G06N 20/00; A61B 5/01

(52) U.S. Cl. CPC A61B 5/7275; A61B 5/01; A61B 5/7267; G06N 20/00

(57) ABSTRACT

A prediction system according to the present application includes an acquisition unit and a prediction unit. The acquisition unit acquires body temperature information indicating a body temperature of a first user and first context information indicating a first context which is a context when the body temperature of the first user is detected. The prediction unit predicts information regarding a body temperature change of a second user in the future, on the basis of a learned model that has learned a relation between the body temperature and the first context and second context information indicating a second context that is a future context of the second user.

### CROSS REFERENCE TO RELATED APPLICATION(S)

The present application claims priority to and incorporates by reference the entire contents of Japanese Patent Application No. 2021-134364 filed in Japan on Aug. 19, 2021.

### BACKGROUND OF THE INVENTION

### 1. Field of the Invention

The present invention relates to a prediction system, a prediction device, a prediction method, and a prediction program.

### 2. Description of the Related Art

Conventionally, a mechanism for predicting a change in a physical condition of a user and providing a prediction result to the user has been proposed. For example, Japanese Unexamined Patent Publication No. 2018-116584 proposes technology for monitoring an activity state and an environmental state of a person, predicting a risk regarding heat stroke, and providing information necessary for heat stroke prevention.

However, in the conventional technology, it is not always possible to cause the user to appropriately grasp what kind of body temperature change occurs according to a future situation.

For example, in the conventional technology, the risk regarding the heat stroke is predicted for each user by using a learning model learned to output the risk of occurrence of the heat stroke when measurement data of the physical condition and measurement data of the environmental state are input as input data.

Further, in the conventional technology, by calculating a prediction value of future measurement data on the basis of a tendency of measurement data in a past certain period, the heat stroke risk may be calculated on the basis of the calculated prediction value.

As described above, since the conventional technology predicts the heat stroke risk of the user from the results of monitoring the activity state and the environmental state of the user, there is no concept of predicting what kind of body temperature change occurs in the future situation of the user.

Therefore, in the conventional technology, there is room for improvement in causing the user to appropriately grasp what kind of body temperature change occurs according to the future situation.

### SUMMARY OF THE INVENTION

It is an object of the present invention to at least partially solve the problems in the conventional technology.

According to one aspect of an embodiment, A prediction system according to the present application includes an acquisition unit and a prediction unit. The acquisition unit acquires body temperature information indicating a body temperature of a first user and first context information indicating a first context which is a context when the body temperature of the first user is detected. The prediction unit predicts information regarding a body temperature change of a second user in the future, on the basis of a learned model that has learned a relation between the body temperature and the first context and second context information indicating a second context that is a future context of the second user.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating an example of a prediction system according to an embodiment;

FIG. 2 is a diagram illustrating an example of information processing according to the embodiment;

FIG. 3 is a diagram illustrating a configuration example of a prediction device according to the embodiment;

FIG. 4 is a diagram illustrating an example of an original data storage unit according to the embodiment;

FIG. 5 is a diagram illustrating an example of a learning data storage unit according to the embodiment;

FIG. 6 is a diagram illustrating an example of a learning result storage unit according to the embodiment;

FIG. 7 is a diagram illustrating an example of a prediction result storage unit according to the embodiment;

FIG. 8 is a diagram illustrating an example of threshold control processing according to the embodiment;

FIG. 9 is a flowchart illustrating a prediction processing procedure according to the embodiment; and

FIG. 10 is a hardware configuration diagram illustrating an example of a computer that implements functions of the prediction device.

### DETAILED DESCRIPTION OF THE PREFERRED EMBODIMENTS

Hereinafter, modes (hereinafter, referred to as “embodiments”) for implementing a prediction system, a prediction device, a prediction method, and a prediction program according to the present application will be described in detail with reference to the drawings. Note that the prediction system, the prediction device, the prediction method, and the prediction program according to the present application are not limited by the embodiments. In the following embodiments, the same parts are denoted by the same reference numerals, and redundant description will be omitted.

### 1. Introduction

Recently, there is an increasing trend to implement measures against infection by heat generation detection. As a result, each individual has been paying attention to his/her body temperature change on a daily basis. For this reason, for example, if it is possible to know in advance what kind of body temperature change occurs according to a future action schedule, it is convenient since an individual can perform various types of health management such as heat stroke countermeasures including infection prevention, but such technology still has room for study.

The present invention has been made in view of the above circumstances, and an object thereof is to provide technology capable of causing a user to appropriately grasp what kind of body temperature change occurs according to a future situation. For this purpose, in the present invention, a relation between a body temperature of the user and a context of the user is learned by machine learning, and what kind of body temperature change occurs according to the future action schedule of the user is predicted on the basis of a learning result. In addition, in the present invention, information regarding a prediction result is provided to the user.

### 2. System

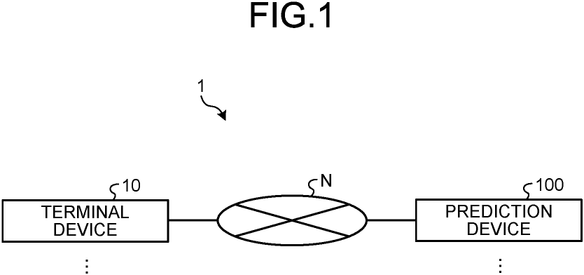

First, a configuration of a prediction system according to an embodiment will be described using FIG. 1. FIG. 1 is a diagram illustrating an example of the prediction system according to the embodiment. FIG. 1 illustrates a prediction system 1 as an example of the prediction system according to the embodiment.

As illustrated in FIG. 1, the prediction system 1 includes a terminal device 10 and a prediction device 100. The terminal device 10 and the prediction device 100 are communicably connected to each other in a wired or wireless manner via a network N. Note that the prediction system 1 illustrated in FIG. 1 may include an arbitrary number of terminal devices 10 and an arbitrary number of prediction devices 100.

The terminal device 10 may be an information processing terminal having an imaging function and a body temperature detection function. The terminal device 10 is realized by, for example, a smartphone, a tablet terminal, a notebook personal computer (PC), a desktop PC, a mobile phone, a personal digital assistant (PDA), or the like.

Further, as described above, the terminal device 10 is not limited to the information processing terminal (possessed by the user) to be daily used by the user, and may be, for example, a monitoring camera for crime prevention installed on a street or the like. Further, as another example, the terminal device 10 may be an information processing terminal for face recognition installed in an entrance gate of a facility or the like for the purpose of authentication for employees or visitors.

In addition, an application for realizing transmission and reception of information to and from the prediction device 100 may be introduced into the terminal device 10. The application may be mounted as a dedicated application for accessing the prediction device 100, or may be a general-purpose application such as a browser.

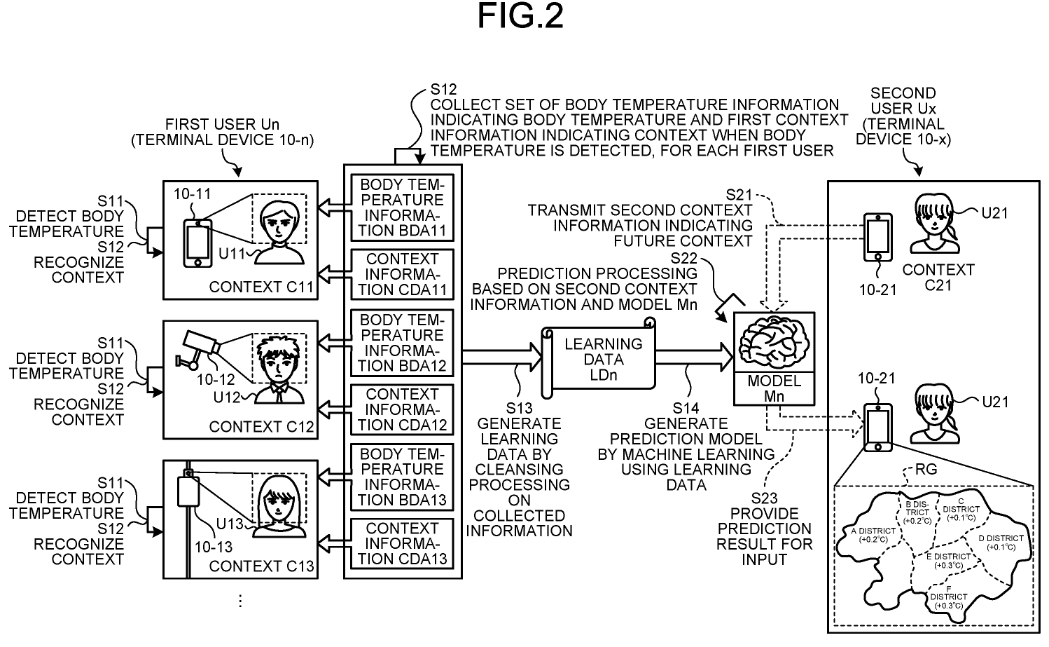

In addition, in the following embodiment, the terminal device 10 may be distinctively expressed according to the user. As illustrated in FIG. 2, for example, the terminal device 10 corresponding to a first user Un who is a user from which original data serving as a source of learning data is acquired is distinctively expressed as a terminal device 10-n (n = 11, 12, 13,...). As a specific example, a terminal device 10-n corresponding to a first user U11 who is an example of the first user Un may be distinctively expressed as a terminal device 10-11.

Further, the terminal device 10 corresponding to a second user Ux who is a processing target user whose information regarding the body temperature change is predicted is distinctively expressed as a terminal device 10-x (x = 21, 22, 23,...). As a specific example, a terminal device 10-x corresponding to a second user U21 who is an example of the second user Ux may be distinctively expressed as a terminal device 10-21.

Note that, in a case where the above-described distinctive notation is unnecessary, the terminal device is simply expressed as the terminal device 10.

The prediction device 100 is an information processing device (server device) that performs, as information processing according to the embodiment, learning processing of generating a model by learning a relation between a body temperature and a context and prediction processing of predicting information regarding a body temperature change using the model in which the relation has been learned. For example, the prediction device 100 can perform such a series of information processing according to a prediction program according to the embodiment.

Further, if the terminal device 10 is used as an edge computer that performs edge processing near the user, the prediction device 100 may be, for example, a cloud computer that performs processing on the cloud side.

Furthermore, the prediction device 100 may be divided into a plurality of devices such as an information processing device that performs learning processing and an information processing device that performs prediction processing. However, in the present embodiment, the prediction device 100 will be described as one information processing device having a function of performing learning processing and a function of performing prediction processing.

Note that, in the example of FIG. 1, an example is illustrated in which the terminal device 10 and the prediction device 100 are individually different devices, but the terminal device 10 and the prediction device 100 may be integrated. As an example of such integration, for example, a function described as being performed by the prediction device 100 in the following embodiment may be mounted on the side of the terminal device 10. As a specific example, a configuration in which the terminal device 10 is operated as the prediction device 100 by introducing the prediction program according to the embodiment into the terminal device 10 may be adopted.

### 3. Specific Example of Information Processing

Next, a specific example of information processing according to the embodiment will be described using FIG. 2. FIG. 2 is a diagram illustrating an example of the information processing according to the embodiment. FIG. 2 illustrates a scene (steps S11 to S14) in which the prediction device 100 performs learning processing of causing the model to learn the body temperature of the first user Un and the first context that is the context when the body temperature is detected by the first user Un. Further, FIG. 2 illustrates a scene (steps S21 to S23) in which the prediction device 100 performs prediction processing of predicting information regarding the body temperature change of the second user Ux in the future, on the basis of the learned model that has learned the relation and the second context that is the future context of the second user Ux.

Hereinafter, as illustrated in FIG. 2, the information processing according to the embodiment will be described by exemplifying first users U11, U12, and U13 as an example of the first user Un and a second user U21 as an example of the second user Ux.

### Learning Processing

Initially, the learning processing will be described. First, the terminal device 10-n detects the body temperature of the first user Un (step S11). According to the example of FIG. 2, an example in which the terminal device 10-11, which is a smartphone, detects the body temperature of the first user U11 is illustrated. Further, according to the example of FIG. 2, an example in which a terminal device 10-12, which is a security camera, detects the body temperature of the first user U12 is illustrated. Furthermore, according to the example of FIG. 2, an example in which a terminal device 10-13, which is an information processing terminal for face recognition, detects the body temperature of the first user U13 is illustrated.

Note that the terminal device 10-n may detect the body temperature of the first user Un on the basis of a surface temperature of the face of the first user Un measured by a thermographic camera. Of course, the terminal device 10-n may measure the body temperature of the first user Un by any other method.

Subsequently, when the terminal device 10-n detects the body temperature of the first user Un, the terminal device 10-n performs processing of recognizing the first context that is the context of the first user Un at the time of detecting the body temperature (step S12). According to the example of FIG. 2, an example in which the terminal device 10-11 recognizes a first context C11 as the context of the first user U11 is illustrated. Further, according to the example of FIG. 2, an example in which the terminal device 10-12 recognizes the first context C12 as the context of the first user U12 is illustrated. Furthermore, according to the example of FIG. 2, an example in which the terminal device 10-13 recognizes a first context C13 as the context of the first user U13 is illustrated.

Note that, although the first context is conceptually expressed in FIG. 2, the first context may be, for example, a position of the first user Un at the time of detecting the body temperature, an action state of the first user Un at the time of detecting the body temperature (for example, an action performed by the first user Un, clothes worn by the first user Un for the action, a congestion situation in which the first user Un is caught as a result of the action, and the like), a surrounding environment of the first user Un at the time of detecting the body temperature (for example, weather, temperature, humidity, and the like), a date and time at the time of detecting the body temperature, an attribute of the first user Un (age, sex, and the like), or the like.

In such a state, the terminal device 10-n transmits, to the prediction device 100, body temperature information indicating the body temperature of the first user Un and first context information indicating a first context that is a context when the body temperature of the first user Un is detected. As a result, as illustrated in FIG. 2, the prediction device 100 collects (acquires), for each first user Un, a set of body temperature information indicating a body temperature and first context information indicating a first context that is a context of the first user Un when the body temperature is detected (step S12).

According to the example of FIG. 2, an example in which the prediction device 100 acquires body temperature information BDA11 as the body temperature information corresponding to the first user U11 and acquires first context information CDA11 as the first context information corresponding to the first user U11 is illustrated. Further, according to the example of FIG. 2, an example in which the prediction device 100 acquires body temperature information BDA12 as the body temperature information corresponding to the first user U12 and acquires first context information CDA12 as the first context information corresponding to the first user U12 is illustrated. Furthermore, according to the example of FIG. 2, an example in which the prediction device 100 acquires body temperature information BDA13 as the body temperature information corresponding to the first user U13 and acquires first context information CDA13 as the first context information corresponding to the first user U13 is illustrated.

Here, the information collected by the prediction device 100 in step S12, that is, the body temperature information indicating the body temperature of the first user Un, and the first context information indicating the first context that is the context when the body temperature of the first user Un is detected are original data serving as a source of learning data used for learning of the model. Therefore, next, the prediction device 100 generates learning data used for model learning by performing cleansing processing on the information collected in step S12 (step S13). According to the example of FIG. 2, the prediction device 100 generates learning data LDn for causing a model Mn to learn a relation between the body temperature change and the context as to what kind of body temperature change tends to occur in the first user Un according to the first context.

For example, the prediction device 100 may generate learning data LD1 configured by a set of information indicating the body temperature change and the first context information as an example of the learning data LDn on the basis of the original data. Further, the prediction device 100 may generate, as another example of the learning data LDn, learning data LD2 configured by a set of probability information indicating a probability that a specific body temperature change will occur according to the first context and the first context information on the basis of the original data.

Next, the prediction device 100 generates a prediction model Mn by causing the model Mn to learn the relation between the body temperature change and the context by machine learning using the learning data LDn (step S14).

For example, by using the learning data LD1 as teacher data, the prediction device 100 generates a first prediction model M1 using context information as input and information indicating the body temperature change as output as the model Mn by machine learning. Further, by using the learning data LD2 as teacher data, the prediction device 100 may generate, as the model Mn, a second prediction model M2 using the context information as input and information indicating what kind of body temperature change occurs with what probability as output.

### Prediction Processing

Next, the prediction processing will be described. First, the terminal device 10-x transmits second context information indicating a second context, which is a future context of the second user Ux, to the prediction device 100 according to the operation by the second user Ux (step S21). According to the example of FIG. 2, an example in which the terminal device 10-21, which is a smartphone, transmits the second context information corresponding to the second user U21 to the prediction device 100 in response to an input operation by the second user U21 is illustrated.

Here, for example, it is assumed that the second user U21 desires to know what kind of body temperature change occurs in second user U21 at 10:00, which is several hours later, in a B district and an area around the B district. In such a case, the second user U21 can input second context information indicating the second context C21 of “scheduled to head for the B district at 10:00” (future action schedule). Further, in such a case, the terminal device 10-21 transmits the second context information indicating the second context C21 of “scheduled to head for the B district at 10:00” to the prediction device 100.

When the prediction device 100 receives the input of the second context information, the prediction device 100 predicts information regarding the body temperature change of the second user U21, on the basis of the received second context information and the model Mn (step S22). For example, the prediction device 100 inputs the second context information to the prediction model M1 and acquires information output from the prediction model M1, that is, the information indicating the body temperature change as a prediction result.

For example, it is assumed that the prediction model M1 outputs “the body temperature change that can occur in a case of being located in the B district at 10:00: +0.2° C.” in response to the input of the second context information. In such a case, the prediction device 100 acquires output information as a prediction result.

In response to the input of “scheduled to head for the B district at 10:00” by the second user U21, the prediction device 100 may dynamically determine second context information in which the second user U21 is assumed to go to another area, thereby further acquiring output information corresponding to another area as the prediction result. For example, the prediction device 100 may dynamically determine the second context information including an area near the B district and an expected area through which the second user U21 is expected to pass when heading from the current location to the B district.

Taking an A district, a C district, a D district, an E district, and an F district as examples of another area, the prediction model M1 outputs the prediction result by inputting the second context information including each of these other districts by the prediction device 100.

Here, it is assumed that the prediction model M1 outputs “the body temperature change that can occur in a case of being located in the A district at 10:00: +0.2° C.”, “the body temperature change that can occur in a case of being located in the C district at 10:00: +0.1° C.”, “the body temperature change that can occur in a case of being located in the D district at 10:00: +0.1° C.”, “the body temperature change that can occur in a case of being located in the E district at 10:00: +0.3° C.”, and “the body temperature change that can occur in a case of being located in the F district at 10:00: +0.3° C.”. In such a\ase, the prediction device 100 can further acquire these five pieces of output information as the prediction result in addition to the prediction result corresponding to the B district.

Note that, by inputting the second context information to the prediction model M2 instead of the prediction model M1, the prediction device 100 may acquire probability information indicating what probability the body temperature change in each district described above occurs with as a prediction result.

Next, the prediction device 100 provides the prediction result acquired in the prediction processing in step S22, that is, the information regarding the body temperature change of the second user U21 to the second user U21 (step S23). For example, the prediction device 100 may generate a prediction result screen RG in which five pieces of output information output by the prediction model M1 have been mapped on a map as the prediction result, and transmit the generated prediction result screen RG to the terminal device 10-21 so as to display the generated prediction result screen RG on the screen.

FIG. 2 illustrates an example of the prediction result screen RG displayed by the terminal device 10-21. According to such an example, a map including the A to F districts is displayed on the prediction result screen RG, and information indicating the body temperature change that can occur in a case of being located in each district at 10:00 is mapped on the map.

As described above with reference to FIGS. 1 and 2, according to the prediction device 100 included in the prediction system 1, the model Mn that has learned the relation between the body temperature of the first user Un and the first context that is the context of the first user Un when the body temperature is detected is generated. Further, according to the prediction device 100, the information regarding the body temperature change of the second user Ux in the future is predicted on the basis of the model Mn and the second context information indicating the second context which is the future context of the second user Ux, and the prediction result is provided to the second user Ux. As a result, the second user Ux can appropriately grasp what kind of body temperature change can occur in the second user Ux according to the future situation (action schedule).

### 4. Configuration of Prediction Device

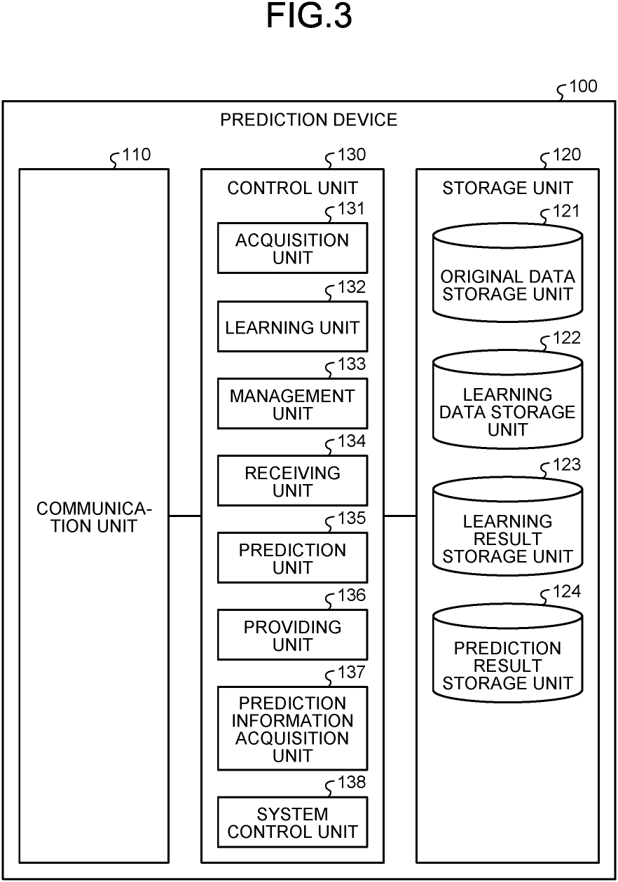

Hereinafter, the prediction device 100 according to the embodiment will be described using FIG. 3. FIG. 3 is a diagram illustrating a configuration example of the prediction device 100 according to the embodiment. As illustrated in FIG. 3, the prediction device 100 includes a communication unit 110, a storage unit 120, and a control unit 130.

### Communication Unit 110

The communication unit 110 is realized by, for example, a network interface card (NIC) or the like. In addition, the communication unit 110 is connected to a network in a wired or wireless manner, and transmits and receives information to and from the terminal device 10, for example.

### Storage Unit 120

The storage unit 120 is realized by, for example, a semiconductor memory element such as a random access memory (RAM) or a flash memory, or a storage device such as a hard disk or an optical disk. The storage unit 120 includes an original data storage unit 121, a learning data storage unit 122, a learning result storage unit 123, and a prediction result storage unit 124.

### Original Data Storage Unit 121

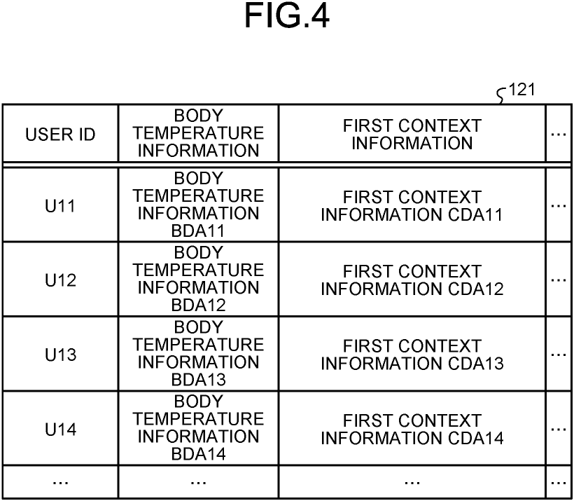

The original data storage unit 121 stores information regarding original data that is a source of learning data. Here, FIG. 4 illustrates an example of the original data storage unit 121 according to the embodiment. In the example of FIG. 4, the original data storage unit 121 has items such as “user ID”, “body temperature information”, and “first context information”.

The “user ID” indicates identification information for identifying the first user Un who is an arbitrary user from which the original data is acquired.

The “body temperature information” is body temperature information indicating the body temperature of the first user Un identified by the “user ID”. As described with reference to FIG. 2, the “body temperature information” may be detected by the terminal device 10-n corresponding to the first user Un. For example, FIG. 4 illustrates an example in which the user ID “U11” and the body temperature information “body temperature information BDA11” are associated with each other. Such an example illustrates an example in which the body temperature of the first user Un (first user U11) identified by the user ID “U11”, that is, the body temperature of “body temperature information BDA11” has been measured by the terminal device 10-11. Note that, in FIG. 4, a conceptual expression such as “body temperature information BDA11” is used, but actually, numerical information indicating the body temperature such as “36.5° C.” is registered.

The “first context information” is information indicating a first context which is a context when the body temperature of the first user Un identified by the “user ID”, that is, the body temperature indicated by the “body temperature information” is detected. For example, FIG. 4 illustrates an example in which the user ID “U11”, the body temperature information “body temperature information BDA11”, and the first context information “first context information CDA11” are associated with each other. Such an example illustrates an example in which the context of the first user U11 when the body temperature indicated by the “body temperature information BDA11” is detected is the first context such as “first context information CDA11”.

### Learning Data Storage Unit 122

The learning data storage unit 122 stores learning data generated from the original data (“body temperature information” and “first context information”) stored in the original data storage unit 121. The learning data is used to cause the model Mn to learn the relation between the body temperature of the first user Un and the first context of the first user Un when the body temperature is detected.

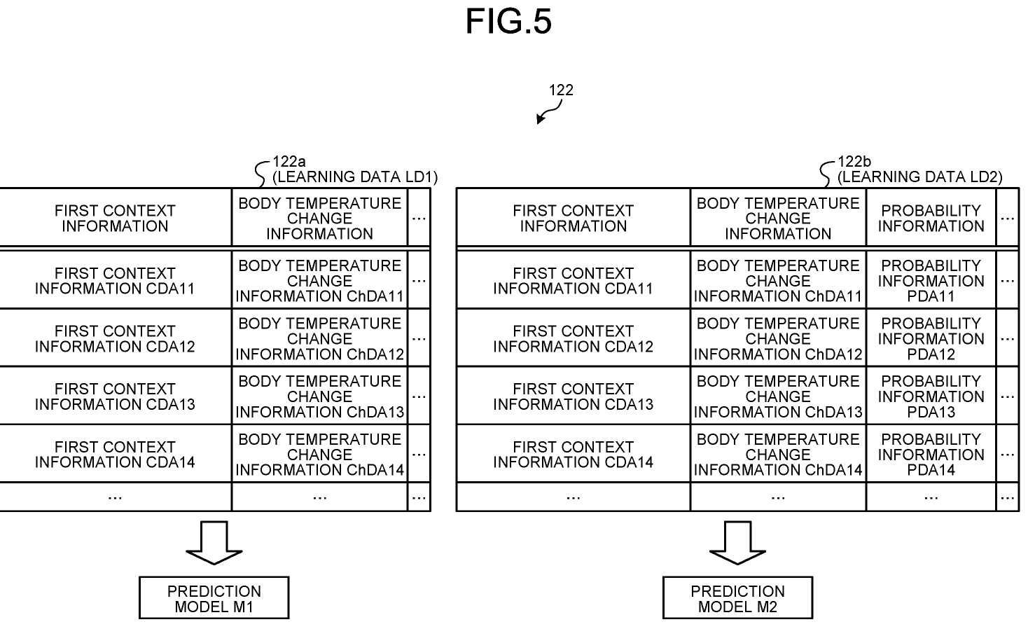

Here, FIG. 5 illustrates an example of the learning data storage unit 122 according to the embodiment. According to the example of FIG. 5, the learning data storage unit 122 may include a learning data storage unit 122a and a learning data storage unit 122b.

For example, the learning data storage unit 122a stores the learning data LD1 including a set of “first context information” and “body temperature change information” that is information indicating a body temperature change that has actually occurred according to the first context indicated by the “first context information”. As described with reference to FIG. 2, the learning data LD1 is used as teacher data for generating the prediction model M1.

For example, FIG. 5 illustrates an example in which the first context information “first context information CDA11” and the body temperature change information “body temperature change information ChDA11” are associated with each other. Such an example illustrates an example in which, in a case of the context indicated by the “first context information CDA11”, results indicating that a body temperature change indicated by the “body temperature change information ChDA11” has occurred in the first user Un are obtained.

In addition, the learning data storage unit 122b stores the learning data LD2 including a set of “first context information”, “body temperature change information” that is information indicating a body temperature change that has actually occurred according to the first context indicated by the “first context information”, and “probability information” indicating a probability that the body temperature change will occur according to the first context. As described with reference to FIG. 2, the learning data LD2 is used as teacher data for generating the prediction model M2.

For example, FIG. 5 illustrates an example in which the first context information “first context information CDA11”, the body temperature change information “body temperature change information ChDA11”, and the probability information “probability information PDA11” are associated with each other. Such an example illustrates an example in which, in a case of the context indicated by the “first context information CDA11”, results indicating that a body temperature change indicated by the “body temperature change information ChDA11” has occurred in the first user Un are obtained. Further, such an example illustrates an example in which a probability that a body temperature change indicated by the “body temperature change information ChDA11” will occur according to the context indicated by the “first context information CDA11” is the “probability information PDA11”.

### Learning Result Storage Unit 123

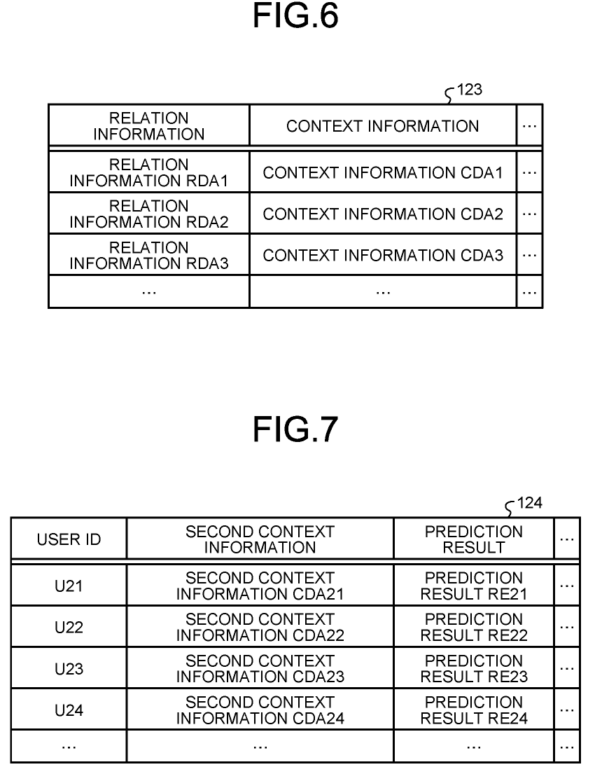

The learning result storage unit 123 stores information regarding the relation learned by the model Mn. Here, FIG. 6 illustrates an example of the learning result storage unit 123 according to the embodiment. In the example of FIG. 6, the learning result storage unit 123 has items such as “relation information” and “context information”.

The “relation information” is information indicating, as a learning result, a relation learned by the model Mn, specifically, a relation between information regarding the body temperature of the first user and the first context when the body temperature of the first user is detected. More specifically, the “relation information” is information indicating a relation between the body temperature change and the context as to what kind of body temperature change tends to occur in the first user Un with what probability according to the first context.

The “context information” indicates future context information estimated from the relation indicated by the “relation information”.

Here, as an example of the relation indicated by the “relation information”, for example, in a first context of “a man in his twenties is caught in congestion at a congestion level LV3 at a point P at 10:00 AM, and the weather at this time is clear/outside temperature 32° C.”, there is a tendency that “the body temperature increases by 0.4° C.”. Further, from the relation, as a future context, “season, weather, temperature, and congestion level at the point P in the future for a predetermined period from a present time point” can be estimated, and an estimation result may be associated with the “relation information” as “context information”.

Further, according to such association, the prediction device 100 can predict the information regarding the body temperature change of the second user without using the model Mn. For example, the prediction device 100 predicts what kind of body temperature change occurs in the second user in the second context indicated by the “second context information”, on the basis of “relation information” associated with “context information” that is matched with (or is similar to) the “second context information” received from the second user in the “context information” of the learning result storage unit 123.

### Prediction Result Storage Unit 124

The prediction result storage unit 124 stores information regarding the body temperature change predicted for the second user as a prediction result. Here, FIG. 7 illustrates an example of the prediction result storage unit 124 according to the embodiment. In the example of FIG. 7, the prediction result storage unit 124 has items such as “user ID”, “second context information”, and “prediction result”.

The “user ID” indicates identification information for identifying the second user Ux who is a processing target user whose information regarding the body temperature change is predicted.

The “second context information” is information indicating a future context of the second user Ux. The “second context information” may be input to the prediction device 100 by the second user Ux identified by the “user ID”, for example. For example, FIG. 7 illustrates an example in which the user ID “U21” and the second context information “second context information CDA21” are associated with each other. Such an example illustrates an example in which the second user Ux (second user U21) identified by the user ID “U21” has inputted the “second context information CDA21” for the purpose of knowing what kind of body temperature change occurs in a case where there is a subsequent change to a state of the context indicated by the “second context information CDA21”.

The “prediction result” indicates a prediction result (information regarding the body temperature change of the second user) predicted when the second context information is input to the model Mn that has learned the relation between the information regarding the body temperature of the first user Un and the first context corresponding to the first user. For example, FIG. 7 illustrates an example in which the user ID “U21”, the second context information “second context information CDA21”, and the prediction result “prediction result RE21” are associated with each other. Such an example illustrates an example in which it is predicted that the body temperature change indicated by the “prediction result RE21” occurs in the second user U21 in a future context indicated by the “second context information CDA21”.

Returning to FIG. 3, the control unit 130 is realized by executing various programs stored in a storage device inside the prediction device 100 using a RAM as a work area by a central processing unit (CPU), a micro processing unit (MPU), or the like. Further, the control unit 130 is realized by, for example, an integrated circuit such as an application specific integrated circuit (ASIC) or a field programmable gate array (FPGA).

As illustrated in FIG. 3, the control unit 130 includes an acquisition unit 131, a learning unit 132, a management unit 133, a receiving unit 134, a prediction unit 135, a providing unit 136, a prediction information acquisition unit 137, and a system control unit 138, and implements or executes a function and an action of information processing described below. Note that an internal configuration of the control unit 130 is not limited to the configuration illustrated in FIG. 3, and may be another configuration as long as information processing to be described later is performed. Further, a connection relation of the processing units included in the control unit 130 is not limited to a connection relation illustrated in FIG. 3, and may be another connection relation.

### Acquisition Unit 131

The acquisition unit 131 acquires body temperature information indicating the body temperature of the first user Un and first context information indicating the first context which is a context when the body temperature of the first user Un is detected. For example, the acquisition unit 131 acquires, as the first context information, first context information indicating a position of the first user Un when the body temperature is detected, an action state of the first user Un when the body temperature is detected, a surrounding environment of the first user Un when the body temperature is detected, a date and time when the body temperature of the first user Un is detected, or an attribute of the first user Un. Further, the acquisition unit 131 registers the acquired body temperature information and the first context information in the original data storage unit 121 in association with each other.

In the example of FIG. 2, the acquisition unit 131 acquires the body temperature information BDA11 as the body temperature information corresponding to the first user U11, and acquires the first context information CDA11 as the first context information corresponding to the first user U11. Further, as illustrated in FIG. 4, the acquisition unit 131 registers the “body temperature information BDA11” and the “first context information CDA11” in the original data storage unit 121 so as to be associated with a user ID “U11” for identifying the first user U11.

### Learning Unit 132

The learning unit 132 causes the model Mn to learn the relation between the information regarding the body temperature of the first user Un and the first context that is the context when the body temperature of the first user Un is detected. For example, the learning unit 132 causes the model Mn to learn the relation between the body temperature change and the context as to what kind of body temperature change tends to occur in the first user Un according to the first context.

As an example, the learning unit 132 may use a set of the information indicating the body temperature change and the first context information as the learning data LD1, and generate, as the model Mn, a first prediction model M1 using the context information as input and the information indicating the body temperature change as output. Further, the learning unit 132 may perform processing of generating the learning data LD1 on the basis of the original data.

As another example, the learning unit 132 may use a set of probability information indicating the probability of occurrence of a specific body temperature change according to the first context and the first context information as the learning data LD2, and generate, as the model Mn, a second prediction model M2 using the context information as input and information indicating what kind of body temperature change occurs with what probability as output. Further, the learning unit 132 may perform processing of generating the learning data LD2 on the basis of the original data.

### Management Unit 133

The management unit 133 manages information indicating the relation learned by the model Mn and context information indicating a future context estimated from the relation in association with each other. For example, when the relation is learned by the model Mn, the management unit 133 estimates the future context according to the relation on the basis of the learned relation.

For example, in a case where the information indicating the relation learned by the model Mn is that “a man in his twenties is caught in congestion at a congestion level LV3 at a point P at 10 :00 AM, and the weather at this time is clear/outside temperature 32° C.”, it indicates a relation between the first context that there is a tendency that “the body temperature increases by 0.4° C.” and the body temperature change. In such a case, the management unit 133 can estimate, for example, “season, weather, temperature, and congestion level at the point P in the future for a predetermined period from a present time point” as a future context on the basis of the above relation. Further, the management unit 133 associates the context information indicating the estimated context with the information indicating the relation, and manages the context information and the information in the learning result storage unit 123.

In addition, the association described above is performed in advance by the management unit 133, so that the prediction unit 135 can predict the information regarding the body temperature change of the second user Ux without using the model Mn. For example, the prediction unit 135 predicts the information regarding the body temperature change of the second user Ux, on the basis of the relation information associated with the context information corresponding to the second context information among the context information managed by the management unit 133.

For example, the prediction unit 135 can predict what kind of body temperature change occurs in the second user Ux in the second context indicated by the “second context information”, on the basis of the “relation information” associated with the “context information” that is matched with (or is similar to) the “second context information” received from the second user among the “context information” of the learning result storage unit 123.

### Receiving Unit 134

The receiving unit 134 receives various types of information. Specifically, the receiving unit 134 receives input information input to the prediction device 100 via the terminal device 10. For example, the receiving unit 134 receives input of second context information indicating a second context that is a future context of the second user Ux.

### Prediction Unit 135

The prediction unit 135 predicts information regarding a future body temperature change of the second user Ux, on the basis of the model Mn that has learned the relation between the body temperature of the first user Un and the first context that is the context when the body temperature of the first user Un is detected and the second context information indicating the second context that is the future context of the second user Ux. For example, the prediction unit 135 predicts what kind of body temperature change occurs in the second user Ux when the scheduled action is actually performed in the future, on the basis of the second context information indicating the action schedule of the second user Ux as the second context information.

Further, the prediction unit 135 predicts the body temperature change occurring in the second user Ux according to the second context, using the first prediction model M1 and the second context information.

Further, the prediction unit 135 may predict the body temperature change occurring in the second user according to the second context and the probability that the body temperature change will occur, using the second prediction model M2 and the second context information.

Further, the prediction unit 135 may predict the information regarding the body temperature change of the second user Ux, on the basis of the relation associated with the context information corresponding to the second context information among the context information managed by the management unit 133.

### Providing Unit 136

The providing unit 136 provides predetermined information to the second user Ux, on the basis of whether or not the prediction result predicted by the prediction unit 135 satisfies a predetermined condition. For example, in a case where a physical risk is predicted in response to the prediction result predicted by the prediction unit satisfying the predetermined condition, the providing unit 136 provides, to the second user Ux, proposal information in which a measure against the predicted risk is proposed. Further, in a case where a physical risk is predicted in response to the prediction result predicted by the prediction unit 135 satisfying the predetermined condition, the providing unit 136 may perform output control such that an alert for warning risk occurrence is output to the second user Ux.

The predetermined condition mentioned here may be an abnormal body temperature (for example, 37.5° C. or higher) suggesting various poor physical conditions or diseases such as heat stroke and infection. Further, the present invention is not limited to this example, and in a case where the second user Ux is predicted to be in a dense state from the prediction result predicted by the prediction unit 135, the providing unit 136 may perform output control such that an alert advising to avoid the dense state is output to the second user Ux.

Further, the prediction unit 135 may specify a plurality of areas according to the second context, on the basis of the second context information received from the second user Ux, and predict information regarding the body temperature change according to the second context for each of the specified areas. In such a case, as described with reference to FIG. 2, the providing unit 136 may generate a prediction result screen RG in which the information regarding the body temperature change predicted for each area has been mapped on a map including the plurality of areas. In addition, the providing unit 136 may transmit the generated prediction result screen RG to the terminal device 10-x of the second user Ux so as to display the screen. According to such information provision, the second user Ux can refer to content such as a body temperature version of a weather forecast of each place.

As a result, for example, the second user Ux can review a future action schedule, take measures such as preparing appropriate possessions according to the action schedule, or change the destination.

### Prediction Information Acquisition Unit 137

In a case where the body temperature of the second user Ux is measured by a health management system that performs health management of the user according to whether or not the body temperature exceeds a predetermined threshold, the prediction information acquisition unit 137 acquires information regarding the body temperature change predicted for the second user Ux.

### System Control Unit 138

The system control unit 138 adjusts the predetermined threshold on the basis of the information acquired by the prediction information acquisition unit 137, and controls the health management system so as to determine a health condition of the second user Ux using the adjusted threshold. For example, the system control unit 138 adjusts the predetermined threshold on the basis of whether or not the body temperature change indicated by the information acquired by the prediction information acquisition unit 137 satisfies a predetermined condition.

For example, when the body temperature change satisfies the predetermined condition, the system control unit 138 determines whether or not a cause satisfying the predetermined condition is a valid cause on the basis of the context information in which the body temperature change is predicted, and adjusts the predetermined threshold in a case where it is determined that the cause is the valid cause. As an example, in a case where it is determined that the cause satisfying the predetermined condition is the valid cause, the system control unit 138 adjusts the threshold to be increased by a value according to the predetermined condition.

### Example of Threshold Control Processing

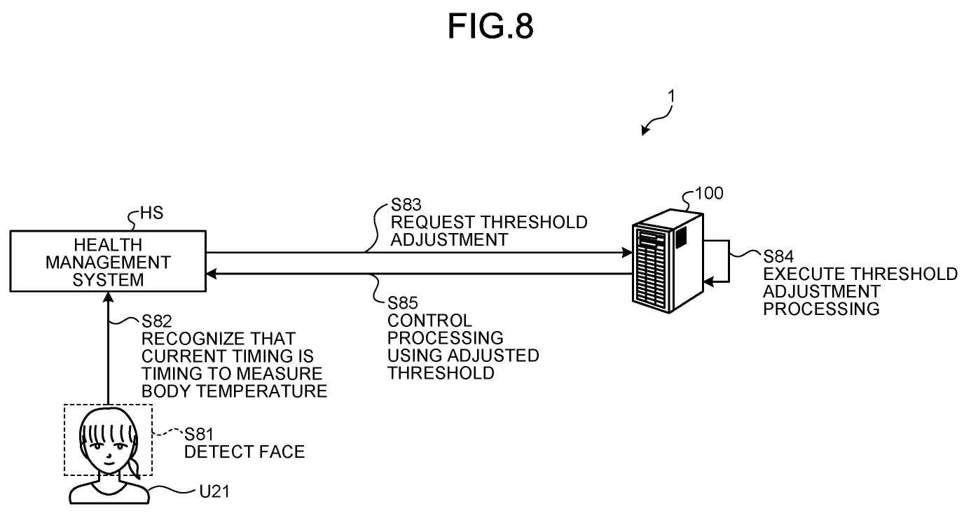

Here, threshold control processing performed by the system control unit 138 will be described using an example of FIG. 8. FIG. 8 is a diagram illustrating an example of threshold control processing according to the embodiment.

FIG. 8 illustrates a scene where a body temperature threshold determined in advance for a health management system HS is adjusted in a case where the body temperature of the second user U21 is measured by the health management system HS. As illustrated in FIG. 8, the health management system HS may be included in the prediction system 1 according to the embodiment. The health management system HS may include an information processing terminal for face recognition installed at an entrance gate of a facility or the like for the purpose of authentication at least for the user. The information processing terminal can detect the body temperature of the user on the basis of a surface temperature of the user measured by the thermographic camera.

In such a state, according to the example of FIG. 8, in response to the face of the second user U21 being detected by the information processing terminal (step S81), the health management system HS recognizes that current timing is timing to measure the body temperature of the second user U21 (step S82).

When it is recognized that the current timing is the timing to measure the body temperature, the health management system HS requests the prediction device 100 to adjust the body temperature threshold (step S83). Such a request may include, for example, notification information indicating the currently set body temperature threshold.

When the prediction device 100 receives a request for threshold adjustment from the health management system HS, the prediction device 100 executes the threshold adjustment processing (step S84). Here, it is assumed that the prediction unit 135 has predicted the body temperature change (for example, an increased temperature relative to an average reference body temperature) of the second user U21 at a time point when the threshold adjustment processing is performed at arbitrary timing in the past. For example, it is assumed that the prediction unit 135 predicts that the body temperature of the second user U21 “increases by 1.0° C.” (body temperature change of 1.0° C.) at the time point when the threshold adjustment processing is performed. As a more specific example, it is assumed that the prediction unit 135 predicts that the body temperature of the second user U21 “increases by 1.0° C.” at the time point when the threshold adjustment processing is performed, on the basis of the second context information indicating the second context of “I will arrive at a company and measure my body temperature immediately after commuting on a crowded train at 8 o’clock” and the prediction model M1.

In the above example, the prediction information acquisition unit 137 acquires a prediction result indicating “body temperature change of 1.0° C.” as information regarding the body temperature change predicted for the second user U21. For example, the prediction information acquisition unit 137 may acquire the prediction result indicating “body temperature change of 1.0° C.” from the prediction result storage unit 124.

In addition, the system control unit 138 controls the body temperature threshold on the basis of the prediction result acquired by the prediction information acquisition unit 137. For example, the system control unit 138 adjusts the body temperature threshold on the basis of whether or not “body temperature change of 1.0° C.” indicated by the prediction result satisfies a predetermined condition. Here, when the abnormal body temperature suggesting various poor physical conditions or diseases such as infection is a “body temperature of 37.5° C. or higher”, a “body temperature change of 1.0° C. or higher” may be set as the predetermined condition on the basis of “1.0° C.” that is a difference between the average reference body temperature of 36.5° C. and the abnormal body temperature of 37.5 of the person.

According to the above example, the system control unit 138 determines that the body temperature change as the prediction result satisfies the predetermined condition by comparing the prediction result “body temperature change of 1.0° C.” acquired by the prediction information acquisition unit 137 with the predetermined condition “body temperature change of 1.0° C. or higher”. As described above, when it is determined that the body temperature change satisfies the predetermined condition, the system control unit 138 determines whether or not a cause satisfying the predetermined condition “body temperature change of 1.0° C. or higher” is a valid cause on the basis of the context information in which the body temperature of the second user U21 is predicted to “increase by 1.0° C.” at a time point when the threshold adjustment processing is performed.

According to the above example, the system control unit 138 can determine whether or not the cause satisfying the predetermined condition “body temperature change of 1.0° C. or higher” is a valid cause on the basis of the second context information indicating the second context of “I will arrive at a company and measure my body temperature immediately after commuting on a crowded train at 8 o’clock”.

For example, even though the user does not suffer from any disease accompanied by a fever, when the user acts in a crowd, performs an abrupt motion, or acts under the blazing sun, the body temperature may temporarily increase. Therefore, if the second context indicated by the second context information corresponds to a situation in which the body temperature is temporarily increased due to a factor other than a disease, the system control unit 138 may determine that the cause satisfying the predetermined condition “body temperature change of 1.0° C. or higher” is a valid cause.

According to the second context mentioned here, the second user U21 measures the body temperature immediately after being caught in a crowd of people such as a crowed train. For this reason, even if the body temperature of the second user U21 increases by “1.0° C. or higher” in spite of not suffering from any disease, it can be determined that an increase in the body temperature is inevitable Therefore, in the example of FIG. 8, the system control unit 138 determines that the cause satisfying the predetermined condition “body temperature change of 1.0° C. or higher” is a valid cause on the basis of the second context information.

In this example in which it is determined that the cause satisfying the predetermined condition “body temperature change of 1.0° C. or higher” is a valid cause, the system control unit 138 adjusts the body temperature threshold to be increased by a value according to the predetermined condition “body temperature change of 1.0° C. or higher”. For example, assuming that the body temperature threshold “37.5° C.” is set in advance according to the abnormal body temperature, the system control unit 138 temporarily adjusts “38.5° C.” obtained by adding “1.0° C.” to the body temperature threshold “37.5° C.” as the body temperature threshold used when the body temperature of the second user U21 is measured this time.

In addition, the system control unit 138 controls the health management system HS to determine the health condition of the second user U21 on the basis of the adjusted body temperature threshold “38.5° C.” (step S85). According to such control processing, the health monitoring system HS measures the body temperature of the second user U21 under the control of the system control unit 138, and determines the health condition of the second user U21 on the basis of the measurement result and the body temperature threshold “38.5° C.”.

On the other hand, when it is determined that the cause satisfying the predetermined condition “body temperature change of 1.0° C. or higher” is not a valid cause (for example, when it is determined that the second user U21 has a fever due to a disease such as infection), the system control unit 138 maintains the current body temperature threshold without adjusting the body temperature threshold. Therefore, in such an example, the system control unit 138 controls the health management system HS so as to determine the health condition of the second user U21 on the basis of the current body temperature threshold “37.5° C.”.

The threshold adjustment processing according to the embodiment has been described using FIG. 8. According to the threshold control processing, for example, it is possible to effectively suppress a situation in which it is erroneously determined that the user suffers from infection accompanied by a fever and the user is unreasonably refused to enter, and it is possible to accurately distinguish a certain user suffering from infection accompanied by a fever. That is, according to such threshold control processing, it is possible to realize the health management system HS that enables effective countermeasures against infection.

### 5. Processing Procedure

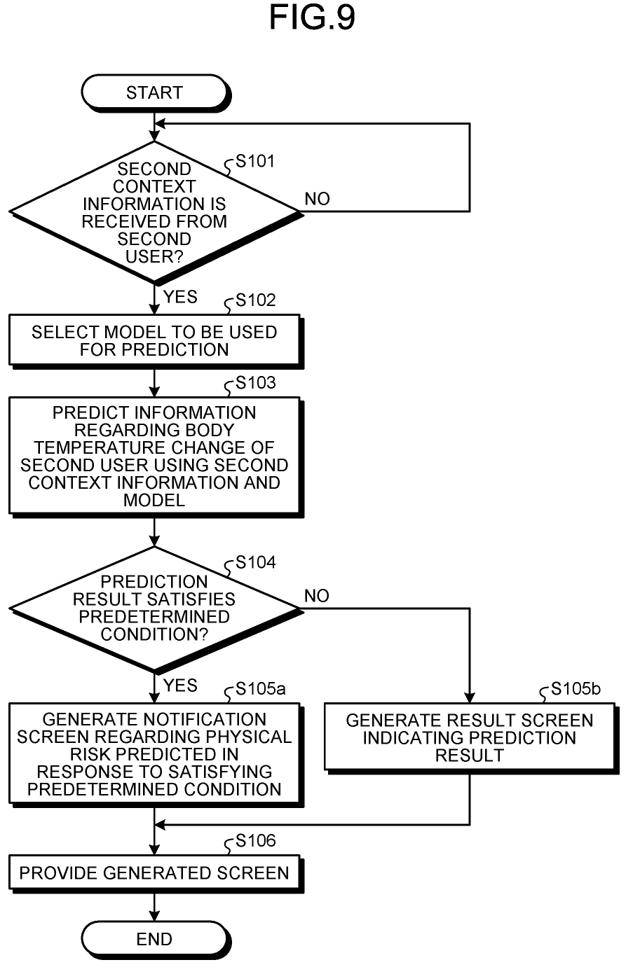

Next, a procedure of the prediction processing according to the embodiment will be described using FIG. 9. FIG. 9 is a flowchart illustrating a prediction processing procedure according to the embodiment. In the example of FIG. 9, the prediction device 100 will be described assuming that the model Mn has been generated by machine learning.

First, the receiving unit 134 determines whether or not the second context information has been received from the second user Ux (step S101). While it is determined that the second context information has not been received (step S101; No), the receiving unit 134 waits until it can be determined that the second context information has been received.

When it is determined that the second context information has been received (step S101; Yes), the prediction unit 135 selects the model Mn used for the prediction processing from the prediction models M1 and M2 (step S102).

Subsequently, using the second context information and the selected model Mn, the prediction unit 135 predicts information regarding the body temperature change of the second user Ux as to what kind of body temperature change occurs according to a future context indicated by the second context information (step S103).

Next, the providing unit 136 determines whether or not a prediction result predicted in step S103 satisfies a predetermined condition (step S104). For example, the providing unit 136 determines whether or not the body temperature of the second user U21 estimated from the predicted body temperature change indicates an abnormal body temperature (for example, 37.5° C. or higher) suggesting various poor physical conditions or diseases such as heat stroke and infection.

Then, when it is determined that the prediction result satisfies the predetermined condition (step S104; Yes), the providing unit 136 generates a notification screen regarding a physical risk predicted in response to the prediction result satisfying the predetermined condition (step S105a). For example, the providing unit 136 may generate a proposal screen on which measures against the physical risk are proposed. Further, for example, the providing unit 136 may generate an alert screen for warning occurrence of the physical risk.

On the other hand, when it is determined that the prediction result does not satisfy the predetermined condition (step S104; No), the providing unit 136 may generate a result screen indicating only the prediction result without including the notification regarding the physical risk (step S105b). For example, the providing unit 136 may generate a result screen indicating information indicating the body temperature change or information indicating the body temperature estimated from the body temperature change.

Further, the providing unit 136 provides the screen generated in step S105a or S105b to the second user Ux (step S106). For example, the providing unit 136 transmits the generated screen to the terminal device 10-x of the second user Ux.

### 6. Other Embodiments

In the above embodiment, an example has been illustrated in which the prediction device 100 causes the model Mn to learn the relation between the information regarding the body temperature of the first user and the first context that is the context when the body temperature of the first user is detected, thereby predicting the information regarding the future body temperature change of the second user using the learned model Mn. Further, an example in which the prediction device 100 provides the prediction result to the second user has been illustrated.

However, instead of providing information only to the second user such as returning the prediction result (information regarding the future body temperature change of the second user) predicted on the basis of the model Mn and the second context information to the second user, according to the request from the second user, as in the above examples, the prediction device 100 may provide information to a specific group. Specifically, the prediction device 100 may provide various types of statistical information calculated from the prediction result to the specific group. As an example of such information provision, the following use cases are considered.

For example, assuming that the specific group is a resident of an area corresponding to a certain municipality M, the prediction device 100 associates a face image of each resident (first user) as a unique ID, and a context (for example, an action log) of the resident of the face image as first context information. In such a case, the prediction device 100 can calculate a regional statistical distribution such as a tendency of a body temperature change in an area unit corresponding to the municipality M on the basis of the first context information, and predict the body temperature change in the area unit from the calculated regional statistical distribution. As a result, the municipality M can provide the prediction result to each resident through a predetermined website, SNS, or the like. In addition to the provision of the prediction result, the municipality M may further provide, for example, a method for improving a lifestyle based on the prediction result, surrounding arousal based on the prediction result, and the like as a resident service for each resident.

The municipality M can also use the prediction result for preparation of a medical system, adjustment of a work shift of a medical worker, replenishment of the medical worker, and the like.

Further, the specific group may be, for example, employees of a private company N other than a resident of an area corresponding to a certain municipality M. In such a case, the company N can use the prediction result for working at home, schedule adjustment, or the like of the employee. The private company N can also use the prediction result for demand prediction, sales promotion, inventory adjustment, production adjustment, appropriate store delivery, and the like of prevention-related products.

### 7. Hardware Configuration

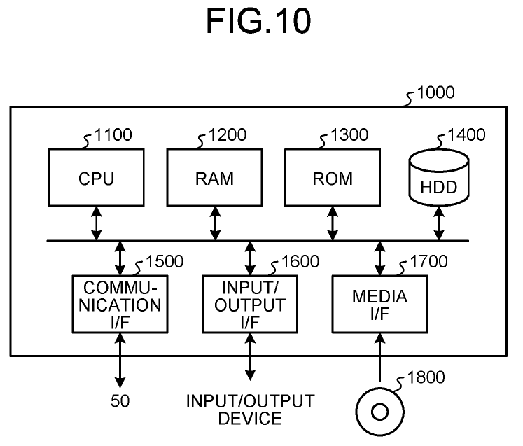

Further, the prediction device 100 described above is realized by a computer 1000 having a configuration illustrated in FIG. 10, for example. FIG. 10 is a hardware configuration diagram illustrating an example of a computer that implements the functions of the prediction device 100. The computer 1000 includes a CPU 1100, a RAM 1200, a ROM 1300, an HDD 1400, a communication interface (I/F) 1500, an input/output interface (I/F) 1600, and a media interface (I/F) 1700.

The CPU 1100 operates on the basis of a program stored in the ROM 1300 or the HDD 1400, and controls each unit. The ROM 1300 stores a boot program executed by the CPU 1100 when the computer 1000 starts, a program depending on hardware of the computer 1000, and the like.

The HDD 1400 stores a program executed by the CPU 1100, data used by the program, and the like. The communication interface 1500 receives data from another device via a predetermined communication network, sends the data to the CPU 1100, and transmits data generated by the CPU 1100 to another device via the predetermined communication network.

The CPU 1100 controls an output device such as a display or a printer and an input device such as a keyboard or a mouse via the input/output interface 1600. The CPU 1100 acquires data from the input device via the input/output interface 1600. Further, the CPU 1100 outputs the generated data to the output device via the input/output interface 1600.

The media interface 1700 reads a program or data stored in a recording medium 1800 and provides the program or data to the CPU 1100 via the RAM 1200. The CPU 1100 loads the program from the recording medium 1800 on the RAM 1200 via the media interface 1700, and executes the loaded program. The recording medium 1800 is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, when the computer 1000 functions as the prediction device 100, the CPU 1100 of the computer 1000 realizes the function of the control unit 130 by executing the program (output control program according to the embodiment) loaded on the RAM 1200. The CPU 1100 of the computer 1000 reads these programs from the recording medium 1800 and executes the programs, but as another example, these programs may be acquired from another device via a predetermined communication network.

### 8. Others

Among the processing described in the above embodiments, all or a part of the processing described as being performed automatically can be performed manually, or all or a part of the processing described as being performed manually can be performed automatically by a known method. In addition, the processing procedures, specific names, and information including various data and parameters illustrated in the document and the drawings can be arbitrarily changed unless otherwise specified. For example, the various types of information illustrated in the respective drawings are not limited to the illustrated information.

In addition, each component of each device illustrated in the drawings is functionally conceptual, and is not necessarily physically configured as illustrated in the drawings. That is, a specific form of distribution and integration of the respective devices is not limited to the illustrated form, and all or a part thereof can be functionally or physically distributed and integrated in an arbitrary unit according to various loads, use situations, and the like.

In addition, the above embodiments can be appropriately combined within a range in which the processing contents do not contradict each other.

Although some of the embodiments of the present application have been described in detail on the basis of the drawings, these are merely examples, and the present invention can be carried out in other forms subjected to various modifications and improvements based on the knowledge of those skilled in the art, including the aspects described in the disclosure of the invention.

In addition, the “part (section, module, or unit)” described above can be replaced with a “mechanism”, a “circuit”, or the like. For example, the acquisition unit can be replaced with an acquisition mechanism or an acquisition circuit.

According to one aspect of an embodiment, it is possible to cause a user to appropriately grasp what kind of body temperature change occurs according to a future situation.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A prediction system comprising:
an acquisition unit that acquires body temperature information indicating a body temperature of a first user and first context information indicating a first context which is a context when the body temperature of the first user is detected; and
a prediction unit that predicts information regarding a body temperature change of a second user in the future, on the basis of a learned model that has learned a relation between the body temperature and the first context and second context information indicating a second context that is a future context of the second user.

2. The prediction system according to claim 1, wherein
the acquisition unit acquires, as the first context information, the first context information indicating a position of the first user when the body temperature is detected, an action state of the first user when the body temperature is detected, a surrounding environment of the first user when the body temperature is detected, a date and time when the body temperature is detected, or an attribute of the first user.

3. The prediction system according to claim 1, wherein
the prediction unit predicts information regarding the body temperature change when a scheduled action is performed in the future, on the basis of second context information indicating an action schedule of the second user as the second context information.

4. The prediction system according to claim 1, further comprising
a learning unit that causes a model to learn a relation between the body temperature and the first context.

5. The prediction system according to claim 4, wherein
the learning unit causes the model to learn a relation between a body temperature change and a context as to what kind of body temperature change tends to occur in the first user according to the first context.

6. The prediction system according to claim 5, wherein
the learning unit uses a set of information indicating the body temperature change and the first context information as learning data and generates, as the model, a first prediction model using context information as input and the information indicating the body temperature change as output, and
the prediction unit predicts a body temperature change occurring in the second user according to the second context, using the first prediction model and the second context information.

7. The prediction system according to claim 5, wherein
the learning unit uses a set of probability information indicating a probability that the body temperature change will occur according to the first context and the first context information as learning data and generates, as the model, a second prediction model using context information as input and information indicating what kind of body temperature change occurs with what probability as output, and
the prediction unit predicts a body temperature change occurring in the second user according to the second context and a probability that the body temperature change will occur, using the second prediction model and the second context information.

8. The prediction system according to claim 5, further comprising
a management unit that manages information indicating a relation learned by the model and context information indicating a future context estimated from the relation in association with each other, wherein
the prediction unit predicts information regarding a body temperature change of the second user, on the basis of a relation associated with context information corresponding to the second context information among context information managed by the management unit.

9. The prediction system according to claim 1, wherein
in a case where the input of the second context information is received from the second user, the prediction unit predicts information regarding a body temperature change of the second user by using the received second context information and the model.

10. The prediction system according to claim 1, further comprising
a providing unit that provides predetermined information to the second user, on the basis of whether or not a prediction result predicted by the prediction unit satisfies a predetermined condition.

11. The prediction system according to claim 10, wherein
in a case where a physical risk is predicted in response to the prediction result predicted by the prediction unit satisfying the predetermined condition, the providing unit provides, to the second user, proposal information in which a measure against the predicted risk is proposed.

12. The prediction system according to claim 10, wherein
in a case where a physical risk is predicted in response to the prediction result predicted by the prediction unit satisfying the predetermined condition, the providing unit outputs an alert for warning occurrence of the risk to the second user.

13. The prediction system according to claim 1, further comprising:
a prediction information acquisition unit that acquires information regarding a body temperature change predicted for the second user in a case where the body temperature of the second user is measured by a health management system that performs health management of a user according to whether or not the body temperature exceeds a predetermined threshold; and
a system control unit that adjusts the predetermined threshold on the basis of the information acquired by the prediction information acquisition unit, and controls the health management system so as to determine a health condition of the second user using the adjusted threshold.

14. The prediction system according to claim 13, wherein
the system control unit adjusts the predetermined threshold on the basis of whether or not a body temperature change indicated by the information acquired by the prediction information acquisition unit satisfies a predetermined condition.

15. The prediction system according to claim 14, wherein
the system control unit determines whether or not a cause satisfying the predetermined condition is a valid cause on the basis of context information in which the body temperature change is predicted in a case where the body temperature change satisfies the predetermined condition, and adjusts the predetermined threshold in a case where it is determined that the cause is the valid cause.

16. The prediction system according to claim 15, wherein
in a case where it is determined that the cause satisfying the predetermined condition is the valid cause, the system control unit adjusts the threshold to be increased by a value according to the predetermined condition.

17. A prediction device comprising:
an acquisition unit that acquires body temperature information indicating a body temperature of a first user and first context information indicating a first context which is a context when the body temperature of the first user is detected; and
a prediction unit that predicts information regarding a body temperature change of a second user in the future, on the basis of a learned model that has learned a relation between the body temperature and the first context and second context information indicating a second context that is a future context of the second user.

18. A prediction method executed by a prediction device, comprising:
acquiring body temperature information indicating a body temperature of a first user and first context information indicating a first context which is a context when the body temperature of the first user is detected; and
predicting information regarding a body temperature change of a second user in the future, on the basis of a learned model that has learned a relation between the body temperature and the first context and second context information indicating a second context that is a future context of the second user.

19. A non-transitory computer-readable storage medium having stored therein a prediction program for causing a prediction device to execute:
acquiring body temperature information indicating a body temperature of a first user and first context information indicating a first context which is a context when the body temperature of the first user is detected; and
predicting information regarding a body temperature change of a second user in the future, on the basis of a learned model that has learned a relation between the body temperature and the first context and second context information indicating a second context that is a future context of the second user.
